(19) United States
(12) Patent Application Publication
(10) Pub. No.: US 2005/0234024 A1
(43) Pub. Date: Oct. 20, 2005

(54) Materials and methods for the treatment of ulcerative colitis

(76) Inventor: David E. Clarke, St. Charles, IL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/823,906

(22) Filed: Apr. 14, 2004

(51) Int. Cl. A61K 31/573; A61K 31/4439; A61K 31/165

(52) U.S. Cl. 514/171; 514/343; 514/617

(57) ABSTRACT

The present invention provides materials and methods for the treatment of inflammatory bowel disease, such as ulcerative colitis, by administering to a patient a nicotine composition comprising an anti-depressant in an amount effective to alleviate symptoms of the disease.

### FIELD OF THE INVENTION

The present invention relates to materials and methods for the treatment of inflammatory bowel disease, such as chronic ulcerative colitis, comprising treatment of affected patients with a combination of nicotine, an analog thereof or a nicotine antagonist and an anti-depressant to ameliorate the symptoms of colitis. The invention also provides materials and methods to administer as a substitute for smoking.

### BACKGROUND OF THE INVENTION

Human ulcerative colitis (UC) is nonspecific idiopathic inflammatory bowel disease (IBD) which forms erosions or ulcers on lamina propria mucosa or submucosa of the large intestine, extending from the rectum to the cecum. Ulcerative colitis (UC) has traditionally been a relatively rare disease affecting primarily non-smokers, however, the number of patients is rapidly increasing in recent years. Ulcerative colitis is predominantly a disease diagnosed at younger ages, for example, most cases are diagnosed before age 30, although the disease can occur at any age. Ulcerative colitis is manifest clinically by symptoms such as diarrhea, bloody stool, abdominal pain and weight reduction, and typically follows a relapsing-remitting disease course.

The cause and morbidity of ulcerative colitis has not been determined to date, but it appears to result from an imbalance in the immune system, wherein the colon and end of the large intestine are chronically inflamed and shed white blood cells (Melgar et al., Clin Exp Immunol. 134:127-37, 2003; Robinson et al., J Lab Clin Med. 130:590-602, 1997). In people with IBD, the immune system mistakes food, bacteria, and other materials in the intestine for foreign or invading substances, and mounts an attack. In the process, the body sends white blood cells into the lining of the intestines, where they produce chronic inflammation. These cells then generate inflammatory cytokines and other harmful products that ultimately lead to ulcerations and bowel injury. The ulcers are tiny open sores which form on the surface of the intestinal lining, where they bleed and produce pus and mucus. When ulcerative colitis affects only the lowest part of the colon, the rectum, it is called ulcerative proctitis. If the disease affects only the left side of the colon, it is called limited or distal colitis. If UC involves the entire colon, it is termed pancolitis.

Approximately half of all patients with ulcerative colitis have relatively mild symptoms. However, others may suffer from severe abdominal cramping, bloody diarrhea, nausea, and fever. The symptoms of ulcerative colitis do enter into periods of remission, which can span months or even years, although symptoms do eventually return. The unpredictable course of ulcerative colitis may make it difficult for physicians to evaluate whether a particular course of treatment has been effective or not. Effective medical treatment can suppress the inflammatory process, permitting the colon to heal and relieving the symptoms of diarrhea, rectal bleeding, and abdominal pain.

Therapies for colitis, such as corticosteroids or aspirin-like aminosalicylates, typically non-specifically target the inflammatory aspect of the disease and provide only temporary relief while having many detrimental side effects. However, recent evidence indicates that non-traditional therapies may have a positive effect at reducing symptoms of ulcerative colitis. For example, studies show that nicotine may have a therapeutic effect at relieving the symptoms associated with ulcerative colitis (Guslandi M., Int J Colorectal Dis. 14:261-262, 1999; Sandbom, W. Am. J. Gastroenter. 94:1161-1171, 1999). It is unclear how nicotine alleviates ulcerative colitis, but may involve nicotinic acetylcholine receptors (nAChRs) in the bowel wall.

Clinical trials have studied the effects of nicotine replacement therapies such as nicotine gum and transdermal nicotine patches on the progression of ulcerative colitis. These studies demonstrated that nicotine administration to patients did have positive effects, but was often very patient dependent and also exhibited side effects to patients not accustomed to nicotine intake (Lashner et al., Dig. Dis. Sci. 35:827-32, 1990; Thomas et al., N. Engl. J. Med. 332:988-92, 1995).

Additional aspects of the smoking cessation market may be contemplated as therapies in other diseases. For example, anti-depressants are commonly used now to assist people to stop smoking. This type of therapy is typically used in place of nicotine replacement therapy. The popular medications ZYBAN® (GlaxoSmithKline) and WELLBUTRIN®, comprised of the anti-depressant burpopion hycrochloride, are useful as short term therapies to aid individuals to quit smoking. Four active metabolites of bupropion, include erythro and threo-amino alcohols erythro-amino diol and morphlinol metabolite-which is rapidly formed. In a newly contemplated therapy, U.S. Pat. No. 6,582,737 contemplates the use of nicotine and bupropion in short term therapy to help patients quit smoking. Warnings for patients undergoing treatment with bupropion (ZYBAN®) indicate the drug may cause gastrointestinal problems at the dosage and combinations currently used.

The cause of ulcerative colitis and the reason for its prevalence in a certain population of patients remains elusive, as do treatments which satisfactorily reduce the severity of the disease without deleterious side effects. Thus, there remains a need in the art to develop a more effective treatment to control the symptoms of ulcerative colitis.

### SUMMARY OF THE INVENTION

The present invention relates to improved materials and methods for the treatment of inflammatory bowel disease, such as ulcerative colitis, and other chronic diseases. In one aspect, the invention provides a method for treating a patient suffering from ulcerative colitis comprising administering a pharmaceutical composition comprising a combination of nicotine, an analog thereof or a nicotine antagonist, and an anti-depressant, wherein the composition is administered in an amount effective to reduce the symptoms of ulcerative colitis. In one embodiment, the anti-depressant is bupropion.

In another aspect, the invention provides materials and methods for the treatment of patients who suffer from a chronic disease in which they can no longer smoke, but cannot alleviate the need for nicotine. In one aspect, it is contemplated that the present invention is used as a substitute for smoking comprising administering to a subject, for a period greater than two months, a composition comprising nicotine, an analog thereof or a nicotine antagonist, and an anti-depressant, wherein the pharmaceutical composition is administered in an amount effective to reduce the patient's need for nicotine. In one embodiment, the anti-depressant is bupropion. In a related embodiment, the subject administered the pharmaceutical composition as a substitute for smoking exhibits a condition selected from the group consisting of inflammatory bowel disease, emphysema, chronic heart failure, lung cancer, and esophageal cancer.

### DETAILED DESCRIPTION OF THE INVENTION

The present invention addresses a need in the art to provide better, more effective treatment for the symptoms of inflammatory bowel disease, such as ulcerative colitis, without the detrimental side effects manifest by current colitis treatments. The present invention provides a method for treating ulcerative colitis using a combination of nicotine and anti-depressant that is suitable for long-term use by a patient.

Nicotine and Nicotine Analogs

In one embodiment, the methods of the invention contemplate treating a patient diagnosed with ulcerative colitis or other chronic disease as set out herein with nicotine or an analog thereof in combination with an anti-depressant. Nicotine and analogs thereof and anti-depressants contemplated by the invention include, but are not limited to, those described below.

A “nicotine composition” as used herein refers to any pharmaceutical formulation of the invention comprising nicotine, an analog thereof or a nicotine antagonist, in combination with any anti-depressant agent set out herein.

Nicotine, a natural agent which has a chemical structure similar to the neurotransmitter acetylcholine, binds to nicotinic aceylcholine receptors (nAchR) in the central nervous system, resulting in stimulation of dopamine release in brainstem pleasure centers. These nAchR are located not only in the brain, but also in other tissues, such as muscles (Tang et al., Cell Signal. 16:551-63, 2004), adrenal glands (Free et al., Brain Res. 974:60-9., 2003) and lymphocytes (Kawashima et al., Life Sci. 72:2101-9, 2003).

To suppress the activity of nicotine on the nAchR, agents that interfere with nicotine binding, or cause altered stimulation of the receptor are used. These agents are useful in therapies to treat nicotine addiction. Nicotine compounds, nicotine derivatives and methods for use have been described in U.S. Pat. No. 4,965,074 to Leeson, which discloses a method for the treatment of memory impairment, especially senile dementia of the Alzheimer's type.

U.S. Pat. No. 5,227,391 to Caldwell et al. discloses a method of treating neuro-degenerative diseases by administering an effective amount of R(+) nicotine. U.S. Pat. No. 5,272,155 to Americ et al. discloses a (+)₂-methylpiperidine which is a specific modulator of the neuronal nicotinic cholinergic receptor and which is useful in the treatment and prevention of cognitive, neurological and mental disorders.

One group of agents used for the suppression of nicotine addiction comprises substances of an alkaloidal nature, such as 1,2,3,4,5,6-hexahydro-1,5-methano-pyrido[1,2-a][1,5]diazocin-8-one (hereafter 'cytisine), lobeline and anabasine hydrochloride, possessing an effect on H-cholinoreactive systems of the organism similar to that of nicotine. The mechanism of their effect is due to their structural similarity with nicotine and the possible “competitive” antagonism between these alkaloids and nicotine (Khalikova et al., Proceedings of Tashkent University, 457: 5-16, 1973).

Description of other nicotine analogs are found in U.S. Pat. No. 6,630,467, which relates to pyridone-fused azabicyclic and cytisine derivatives, and U.S. Pat. No. 5,977,131 which relates to azaindole amine compounds.

A current first line therapy for smoking cessation, as described in U.S. Pat. No. 5,016,652, involves use of a transdermal patch comprising nicotine as a method for the controlled delivery of nicotine to the bloodstream of the user, thereby reducing the incidence of smoking. Also available are nicotine containing gums (U.S. Pat. No. 6,344,222) nicotine lozenges (U.S. Pat. No. 6,280,761), and nicotine inhalation sprays (U.S. Pat. No. 6,596,740). Another common therapy to aid patients in quitting smoking relies on use of an anti-depressant, e.g. an opiod antagonist (U.S. Pat. No. 6,004,970), a tricyclic antidepressant, such as imipramine (U.S. Pat. No. 4,788,189), a serotonin re-uptake inhibitor (U.S. Pat. No. 6,677,378), or an anti-depressant of the aminoketone class, such as bupropion (U.S. Pat. No. 6,495,605).

Bupropion is usually taken in oral form and undergoes a very rapid first pass metabolism. Following oral dosage, blood concentrations of the drug is dose proportional from 100 to 250 mgs. The concentration ‘C’ max occurs in approximately 2 hours, and bupropion's biological half life is approximately 14 hours (range 8-24 hours) (U.S. Pat. No. 6,280,763).

Additional antidepressants useful for combination with nicotine or nicotine analog or substitute include, but are not limited to the following compounds: Adatanserin Hydrochloride; Adinazolam; Adinazolam Mesylate; Alaproclate; Aletamine Hydrochloride; Amedalin Hydrochloride; Amitriptyline Hydrochloride; Amoxapine; Aptazapine Maleate; Azaloxan Fumarate; Azepindole; Azipramine Hydrochloride; Bipenamol Hydrochloride; Bupropion Hydrochloride; Butacetin; Butriptyline Hydrochloride; Caroxazone; Cartazolate; Ciclazindol; Cidoxepin Hydrochloride; Cilobamine Mesylate; Clodazon Hydrochloride; Clomipramine Hydrochloride; Cotinine Fumarate; Cyclindole; Cypenamine Hydrochloride; Cyprolidol Hydrochloride; Cyproximide; Daledalin Tosylate; Dapoxetine Hydrochloride; Dazadrol Maleate; Dazepinil Hydrochloride; Desipramine Hydrochloride; Dexamisole; Deximafen; Dibenzepin Hydrochloride; Dioxadrol Hydrochloride; Dothiepin Hydrochloride; Doxepin Hydrochloride; Duloxetine Hydrochloride; Eclanamine Maleate; Encyprate; Etoperidone Hydrochloride; Fantridone Hydrochloride; Fenmetozole Hydrochloride; Fenmetramide; Fezolamine Fumarate; Fluotracen Hydrochloride; Fluoxetine; Fluoxetine Hydrochloride; Fluparoxan Hydrochloride; Gamfexine; Guanoxyfen Sulfate; Imafen Hydrochloride; Imiloxan Hydrochloride; Imipramine Hydrochloride; Indeloxazine Hydrochloride; Intriptyline Hydrochloride; Iprindole; Isocarboxazid; Ketipramine Fumarate; Lofepramine Hydrochloride; Lortalamine; Maprotiline; Maprotiline Hydrochloride; Melitracen Hydrochloride; Milacemide Hydrochloride; Minaprine Hydrochloride; Mirtazapine; Moclobemide; Modaline Sulfate; Napactadine Hydrochloride; Napamezole Hydrochloride; Nefazodone Hydrochloride; Nisoxetine; Nitrafudam Hydrochloride; Nomifensine Maleate; Nortriptyline Hydrochloride; Octriptyline Phosphate; Opipramol Hydrochloride; Oxaprotiline Hydrochloride; Oxypertine; Paroxetine; Phenelzine Sulfate; Pirandamine Hydrochloride; Pizotyline; Pridefine Hydrochloride; Prolintane Hydrochloride; Protriptyline Hydrochloride; Quipazine Maleate; Rolicyprine; Seproxetine Hydrochloride; Sertraline Hydrochloride; Sibutramine Hydrochloride; Sulpiride; Suritozole; Tametraline Hydrochloride; Tampramine Fumarate; Tandamine Hydrochloride; Thiazesim Hydrochloride; Thozalinone; Tomoxetine Hydrochloride; Trazodone Hydrochloride; Trebenzomine Hydrochloride; Trimipramine; Trimipramine Maleate; Venlafaxine Hydrochloride; Viloxazine Hydrochloride; Zimeldine Hydrochloride; Zometapine (See U.S. Pat. No. 5,780,051).

Treatment of Ulcerative Colitis

In one embodiment of the invention, it is contemplated that the nicotine composition and anti-depressant are given in conjunction with a second agent, wherein the second agent may be an additional ulcerative colitis treatment as described below. It is contemplated that the additional ulcerative colitis treatment is selected from the group consisting of an aminosalicylate, a corticosteroid, an immunomodulatory medicine, a steroid hormone, and a probiotic therapy. In a related embodiment, the ulcerative colitis treatment is mesalazine, salazosulfapyridine, or budesonide.

The first symptom of ulcerative colitis is a progressive loosening of the stool. The stool is generally bloody and may be associated with crampy, abdominal pain and severe urgency to have a bowel movement. The diarrhea may begin slowly or quite suddenly. Loss of appetite and subsequent weight loss are common, as is fatigue. In cases of severe bleeding, anemia may also occur. In addition, there may be skin lesions, joint pain, eye inflammation, and liver disorders. Children with ulcerative colitis may fail to develop or grow properly.

Physicians base a diagnosis of ulcerative colitis based in part on the patient's clinical history, and a physical examination of the rectal area. Stool specimens are obtained and analyzed to eliminate the possibility of bacterial, viral, or parasitic causes of diarrhea. Blood tests can check for signs of infection as well as for anemia, which may indicate bleeding in the colon or rectum. Following this, the patient generally undergoes an evaluation of the colon, using either a sigmoidoscopy or total colonoscopy. Clinical remission is evaluated by Rachmilewitz's activity index (Rachmilewitz, D., Brit. Med. J 298:82-86, 1989), a variation thereof, or other appropriate activity index (Li et al., Curr. Gastroenterol. Rep. 4:490-96, 2002), and confirmed by sigmoidoscopy. The disease activity index is measured as a combined score of decrease in body weight, mucosal appearance, presence of blood in stool, histological indications, and extraintestinal symptoms such as fever.

For example, disease activity in UC patients is assessed by using the colitis activity index (CAI), adapted from Rachmilewitz (Lichtiger et al., N Engl J. Med. 330:1841-1845, 1994). The CAI includes 6 symptoms: general well-being, abdominal tenderness, pain or cramping, fecal incontinence, daily and nocturnal diarrhea, and visible blood in stool. This was chosen because it relies entirely on symptoms and does not require invasive procedures. Total scores range from 0 to 21, with scores less than 10 indicating remission. Patients are classified as having ‘no or minor symptoms’ (CAI score less than 10) and ‘severe symptoms’ (CAI more than 10)

Classes of medication used today to treat ulcerative colitis include aminosalicylates, corticosteroids, and immunomodulatory medicines. Aminosalicylates include aspirin-like drugs that contain 5-aminosalicylic acid (5-ASA). Examples are mesalamine (mesalazine), olsalazine, and sulfasalazine. Balsalazide (Colazal™) is a recently developed oral medication that is specifically designed to treat mild to moderate active ulcerative colitis. It has been shown to be highly useful in treating patients who failed mesalamine therapy. These drugs, which can be given either orally or rectally, alter the body's ability to generate and sustain inflammation. Without inflammation, symptoms such as diarrhea, rectal bleeding, and abdominal pain can be diminished greatly. Aminosalicylates are effective in treating mild to moderate episodes of ulcerative colitis, and are also useful in preventing relapses of this disease.

In clinical treatments for ulcerative colitis, steroid hormone, Salazosulfapyridine (SASP) [SALAZOPYRIN®] and metronidazole [FLAGYL®] are mainly used [New Engl. J. Med., 305:1569-70, 1981; The Merck Manual, Seventeenth Edition, p. 309, 1999]. SASP, which is an azo compound of 5-aminosalicylic acid (5-ASA) and sulfapyridine, is used as the first choice drug particularly for active ulcerative colitis at a minor to moderate stage, and is effective only when a lesion is present in the large intestine. Its effect is relatively weak at a severe stage, and it is in many cases used together with another agent such as a steroid drug even at a minor stage. Further, it is also pointed out that the effect is insufficient at an acute stage of inflammation. Its detailed mechanism of action is still unclear, even though its various actions have been reported, such as prostaglandin synthesis inhibitory action, leukotriene synthesis inhibitory action, leukocyte chemotaxis inhibitory action, oxygen radical production inhibitory and erasing action, immunosuppressive action and anti-inflammatory action. Further, adverse reactions to SASP include liver function failure, nausea and vomiting, headache, pyrexia, hemolytic anemia, male sterility, abdominal dysphoria, rash, lymph node swelling, granulocytopenia and folic acid deficiency appear, and the frequency reaches 10 to 20% (Gastrointestinal Pharmacology, 21:643-658, 1992).

Mesalazine, which is a sustained release preparation coated so that 5-ASA is formed at the local pH in the intestine, has been developed and used clinically in order to decrease the adverse reactions of SASP, but side-effects similar to those of SASP have been reported, and its effect does not exceed SASP [Japanese Pharmacology & Therapeutics, 22:93-121 (1994)]. Additionally, adreno-corticosteroids such as Predonine or Rinderon have commonly been used to treat UC, but have other adverse reactions due to virus and bacterial infection or suppression of pituitary gland and adrenal cortex function [Sogo Rinsho (Comprehensive Clinic), 43:1725-1729 (1994)].

Corticosteroids include prednisone, methylprednisolone, and budesonide. These medications can be given orally, rectally, or intravenously. Corticosteroids are used for patients with moderate to severe disease. These drugs affect the body's ability to create and maintain inflammation. Although steroids can be quite effective for short-term control of acute episodes of colitis (flare-ups), they are not recommended for long-term use due to side effects. For example, osteoporosis (bone loss), cataracts, stretch marks, weight gain, diabetes, hypertension and psychiatric symptoms frequently occur after long-term use. Thus, doctors often choose safer medications (such as mesalamine products or antibiotics) as initial therapy.

Budesonide (ENTOCIRT™ EC, AstraZeneca) received FDA approval in 2001, and represents a new class of corticosteroids: nonsystemic steroids. This type of drug is rapidly metabolized and quickly cleared from the bloodstream. As a result, it substantially reduces side effects.

Immunomodulatory medicines include azathioprine, 6-mercaptopurine (6-MP), 6 thioguanine, (6-TG) and, recently, cyclosporine. These therapies alter the immune cells' interaction with the inflammatory process. Immunomodulators are generally administered orally. They are used in selected patients when aminosalicylates and corticosteroids have been either ineffective or only partially effective. Azathioprine and 6-MP have been useful in reducing or eliminating some patients' dependence on corticosteroids. They also may be helpful in maintaining remission in selected refractory ulcerative colitis patients (that is, patients who do not respond to standard medications). However, these medications can take as long as three months before their beneficial effects begin to work.

A more extreme, but often seen treatment of colitis is surgery. Approximately 25 to 40 percent of people with ulcerative colitis will require surgery at some time during their illness. Over the past decade, surgery in ulcerative colitis has evolved to a degree that offers several alternatives to achieve both a better quality of life and a cure for disease. The most recommended procedure is proctocolectomy (removal of the colon and rectum). Prior to 1980, this involved placement of a permanent ileostomy. Currently, the ileoanal pouch anal anastomosis (IPAA) or restorative proctocolectomy is an option for many people.

Another treatment being attempted in ulcerative colitis is the use of probiotic therapies, wherein intestinal bacteria are administered to the patient in an effort to restore the natural flora of the intestinal tract and relieve symptoms of ulcerative colitis (Tamboli et al, Best Pract Res Clin Gastroenterol. 17:805-20, 2003).

Formulation of Pharmaceutical Compositions

The methods of the invention are preferably carried out using a nicotine composition as described herein with one or more pharmaceutically acceptable carriers. The phrase “pharmaceutically or pharmacologically acceptable” refer to molecular entities and compositions that do not produce any allergic, or other adverse reactions when administered using routes well-known in the art, and described below. “Pharmaceutically acceptable carriers” also include any and all clinically useful solvents, dispersion media, coatings, antibacterial and antifungal agents, isotonic and absorption delaying agents and the like. The use of such media and agents for pharmaceutically active substances is well known in the art.

Pharmaceutical carriers include pharmaceutically acceptable salts, particularly where a basic or acidic group is present in a compound. For example, when an acidic substituent, such as —COOH, is present, the ammonium, sodium, potassium, calcium and the like salts, are contemplated for administration. Additionally, where an acid group is present, pharmaceutically acceptable esters of the compound (e.g., methyl, tert-butyl, pivaloyloxymethyl, succinyl, and the like) are contemplated as preferred forms of the compounds, such esters being known in the art for modifying solubility and/or hydrolysis characteristics for use as sustained release or prodrug formulations.

When a basic group (such as amino or a basic heteroaryl radical, such as pyridyl) is present, then an acidic salt, such as hydrochloride, hydrobromide, acetate, maleate, pamoate, phosphate, methanesulfonate, p-toluenesulfonate, and the like, is contemplated as a form for administration.

In addition, compounds may form solvates with water or common organic solvents. Such solvates are contemplated as well.

The nicotine compositions may be administered orally, topically, transdermally, parenterally, rectally, or by injection. The term parenteral as used herein includes subcutaneous injections, intravenous, intramuscular, or intracisternal injection, or infusion techniques. Administration by intravenous, intradermal, intramusclar injection and or surgical implantation at a particular site is contemplated as well. Generally, compositions are essentially free of pyrogens, as well as other impurities that could be harmful to the recipient.

The pharmaceutical compositions containing nicotine as described above are in a form suitable for oral use, for example, as tablets, troches, lozenges, aqueous or oily suspensions, dispersible powders or granules, emulsions, hard or soft capsules, or syrups or elixirs. Compositions intended for oral use are prepared according to any known method, and such compositions contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients can include, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the mouth or gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for controlled release.

Formulations for oral use may be presented as hard gelatin capsules wherein the active ingredient is mixed with an inert solid diluent, for example, calcium carbonate, calcium phosphate or kaolin, or as soft gelating capsules wherein the active ingredient is mixed with water or an oil medium, for example peanut oil, liquid paraffin, or olive oil.

Aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

The pharmaceutical compositions of the invention may also be in the form of oil-in-water emulsions. The oily phase may be a vegetable oil, for example olive oil, arachis oil, sesame oil or coconut oil, or a mineral oil, for example liquid paraffin, or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

Transdermal delivery of drugs can be accomplished from topical products such as ointments or cremes or from transdermal devices (See U.S. Pat. No. 6,620,428). A transdermal device includes a transdermal patch. Devices usable as transdermal patches can be categorized in several different ways. See Steven Wick, “Developing A Drug-In-Adhesive Design For Transdermal Drug Delivery”, Adhesives Age, 1995; 38(10): 18-24, which hereby is incorporated by reference. Transdermal devices are divided into the following four main groups: the reservoir type, in which the drug is placed in a liquid or a gel and is delivered to the skin across a rate-moderating membrane; the matrix type, in which the drug is placed within a non-adhesive polymeric material, typically a hydrogel or soft polymer; the drug-in-adhesive type, in which the drug is placed within an adhesive polymer; and the multi-laminate type, which is similar to the drug-in-adhesive design, but which incorporates an additional layer of pressure sensitive adhesive to cover the entire device and affix it to the skin. A fifth type is the iontophoretic type, in which an electrical potential gradient is used for transferring the drug through the skin—see e.g. Parminder Singh et al, “Iontophoresis in Drug Delivery: Basic Principles and Applications”, Critical Reviews in Therapeutic Drug Carrier Systems, 11: 161-213, 1994.

The liquid or gel used in the reservoir type device could be hydrophilic or lipophilic, such as water, alcohols, mineral oils, silicone fluids, various copolymers, such as ethylene vinyl acetate, vinyl acetate or polyvinyl alcohol/polyvinyl pyrrolidine. The reservoir may also include dyes, inert fillers, diluents, antioxidants, penetration enhancers, stabilizers, solubilizing agents and other pharmacologically inactive pharmaceutical agents being well known in the art.

The adhesives used are generally of three types, being the rubber type, for example polyisobutylenes, the acrylate type and the silicone type. The adhesives may be chemically modified and may have a wide range of molecular weights. Excipients such as fillers, stabilizers, plasticizers, buffering agents, penetration enhancers, penetration retarders, solubilizing agents and other pharmaceutical ingredients being well known in the art may be added to the adhesive.

Polymer films which may be used for making the rate-moderating membrane include, without limitation, those comprising low density polyethylene, high density polyethylene, ethyl vinyl acetate copolymers and other suitable polymers.

The backing layer serves the purposes of preventing passage of the drug or environmental moisture through the surface of the patch distant from the skin (skin-distal), and also for providing support for the system, where needed. The backing layer may be chosen so that the end product is appealing to the users, whether children, adults, elderly people or other customer groups. The backing layer is impermeable (occlusive) to the passage of nicotine compositions or inactive ingredients being present in the formulation and can be flexible or nonflexible. Suitable materials include, without limitation, polyester, polyethylene terephthalate, some types of nylon, polypropylene, metallized polyester films, polyvinylidene chloride and aluminium foil. The layer is preferably in the range of about 15 micrometers to about 250 micrometers in thickness. A release liner can be made of the same materials as the backing layer.

The transdermal route of parenteral delivery of drugs, other biologically active agents, or pharmaceutical composition of the invention, on either a rate-controlled or non-rate-controlled basis is described in numerous publications, such U.S. Pat. Nos. 3,598,122; 5,342,623; 5,635,203, and 6,699,497, the disclosures of which are incorporated in their entirety herein by reference. Some factors to be considered in the formulation of a transdermal patch are skin permeability and skin binding. The amount of composition bound should be supplied to the skin and delivered into the blood stream at steady, therapeutically effective rates. If large amounts of the agent are bound in the skin, significant delays in the onset of therapeutic effect (“lag time”) will be observed. Additionally, skin irritation and sensitivity is considered. Many topically applied substances can cause blistering or reddening accompanied by unpleasant burning, itching, and stinging sensations. Animal models are used to screen for irritation. Animal models, however, often produce both false positives and false negatives. There is also a wide interpersonal variation in susceptibility to irritation. An agent must be minimally irritating in a large percentage of the target population in order to be suitable for safe and effective transdermal administration. U.S. Pat. Nos. 4,552,872, 4,756,710, 5,028,431, 5,130,139, 5,160,741, 5,304,379, and 5,451,407 are directed to overcoming problems of skin irritation associated with transdermal drug delivery and are hereby incorporated in their entirety by reference. Further, pharmokinetic properties, potency and metabolism of the agent must be considered.

A surface for transdermal administration, wherein the area covered by the patch may be in the range of between about five to forty square centimeters, typically comprises a reservoir (depot layer) to hold the agent to be administered and a permeation enhancer; a backing behind the body contacting-distal surface of the reservoir. The permeation enhancer may be any permeation enhancer known in the art to increase permeability of drugs through skin and includes, but is not limited to, those disclosed in the above cited patents. In one aspect, the permeation enhancer is a monoglyceride, a C₁₀-C₂₀ fatty acid ester, including ethyl palmitate and isopropyl myristate; acyl lactylates such as caproyl lactylic acid and lauroyl lactylic acid; dimethyl lauramide; dodecyl (lauryl) acetate; a lactate ester such as lauryl lactate, and myristyl lactate; a monoalkyl ether of polyethyleneglycol and their alkyl or aryl carboxylic acid esters and a carboxymethyl ether such as polyethylene glycol-4 lauryl ether (Laureth-4) and polyethylene glycol-2 lauryl ether (Laureth-2); Myreth-3, myristyl sarcosine, and methyl laurate.

In one embodiment, the transdermal administration of nicotine composition is obtained by administering a transdermal system comprising: (a) a nicotine composition depot layer, having a skin-facing side and a skin-distal side, the depot layer containing a sufficient quantity of nicotine composition to maintain a useful flow of nicotine from the patch for a total time period of 12-16 hours or more; (b) an occlusive backing layer in contact with and covering the depot layer on the skin-distal side; and (c) rate-controlling means for controlling diffusion of nicotine composition from the skin-facing side. In a related embodiment, the rate controlling means permits a first flow of greater than zero but less than 2 mg/cm² nicotine composition in any hour for a first time period of greater than zero but less than 5 hours, then a second flux between 20 and 800 μg/cm²/h for a second time period of 7 hours or more, wherein the transdermal system results in nicotine blood levels of between 5 to 30 ng/mL for at least 12 hours.

The pharmaceutical compositions may be in the form of a sterile injectable aqueous, oleaginous suspension, dispersions or sterile powders for the extemporaneous preparation of sterile injectable solutions or dispersions. The suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. The carrier can be a solvent or dispersion medium containing, for example, water, ethanol, polyol (for example, glycerol, propylene glycol, and liquid polyethylene glycol, and the like), suitable mixtures thereof, vegetable oils, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

For easy syringability, the form must be sterile and must be fluid. The proper fluidity can be maintained, for example, by the use of a coating, such as lecithin, by the maintenance of the required particle size in the case of dispersion and by the use of surfactants. It must be stable under the conditions of manufacture and storage and must be preserved against the contaminating action of microorganisms, such as bacteria and fungi. The prevention of the action of microorganisms can be brought about by various antibacterial an antifungal agents, for example, parabens, chlorobutanol, phenol, sorbic acid, thimerosal, and the like. In many cases, it will be desirable to include isotonic agents, for example, sugars or sodium chloride. Prolonged absorption of the injectable compositions can be brought about by the use in the compositions of agents delaying absorption, for example, aluminum monostearate and gelatin.

The compositions may also be in the form of suppositories for rectal administration of the nicotine composition. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols, for example.

Compositions useful for administration may be formulated with uptake or absorption enhancers to increase their efficacy. Such enhancer include for example, salicylate, glycocholate/linoleate, glycholate, aprotinin, bacitracin, SDS, caprate and the like. See, e.g., Fix (J. Pharm. Sci., 85:1282-1285, 1996) and Oliyai and Stella (Ann. Rev. Pharmacol. Toxicol., 32:521-544, 1993).

Nicotine compositions contemplated for use relieve symptoms associated with ulcerative colitis, including inflammation, rectal irritation and bleeding, thereby rendering them particularly desirable for the treatment of ulcerative colitis. In particular, the compositions exhibit beneficial effects at concentrations that are substantially free of side effects, and are therefore useful for extended treatment protocols. For example, co-administration of a nicotine composition with another, anti-inflammatory agent can achieve beneficial inhibition of a ulcerative colitis, while effectively reducing the toxic side effects in the patient.

In addition, the properties of hydrophilicity and hydrophobicity of the compositions contemplated for use in the invention are well balanced, thereby enhancing their utility, while other compositions lacking such balance are of substantially less utility. Specifically, compositions contemplated for use in the invention have an appropriate degree of solubility in aqueous media which permits absorption and bioavailability in the body, while also having a degree of solubility in lipids which permits the compounds to traverse the cell membrane to a putative site of action.

Administration and Dosing

The nicotine composition of the invention may be administered to a patient suffering from ulcerative colitis or other chronic disease as described above in any manner determined to be appropriate by the treating physician.

In one aspect, methods of the invention include a step of administration of a pharmaceutical nicotine composition comprising nicotine or an analog thereof and an anti-depressant in an amount effective to alleviate symptoms of ulcerative colitis, or alleviate the need for smoking in patients exhibiting a chronic disease preventing intake of nicotine via smoking. A “therapeutically effective amount” or “effective amount” refers to that amount of the compound sufficient to result in amelioration of symptoms, for example, treatment, healing, prevention or amelioration of the relevant medical condition, or an increase in rate of treatment, healing, prevention or amelioration of such conditions. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

Methods of the invention are performed using any medically-accepted means for introducing a therapeutic composition directly or indirectly into a mammalian subject, including but not limited to injections, oral ingestion, intranasal, topical, transdermal, parenteral, inhalation spray, or rectal administration. The term parenteral as used herein includes subcutaneous, intravenous, intramuscular, and intracistemal injections, as well as catheter or infusion techniques. Administration by, intradermal, intramammary, intraperitoneal, intrathecal, retrobulbar, intrapulmonary injection and or surgical implantation at a particular site is contemplated as well.

In one embodiment, administration is performed at the site of the ulcerative colitis lesion or affected tissue needing treatment by direct topical administration via liquid enema at the site or via a sustained delivery or sustained release mechanism, which can deliver the formulation internally. In another embodiment, the composition is administered transdermally in patch form, and may be placed on the body at an appropriate location, preferably on the trunk of the body. In another embodiment, the composition is administered orally. In yet another embodiment, the composition is administered rectally, either in fluid enema or suppository form.

Therapeutic compositions may also be delivered to the patient at multiple sites. The multiple administrations may be rendered simultaneously or may be administered over a period of time. Additional therapy may be administered on a period basis, for example, hourly, daily, weekly or monthly.

Particularly contemplated in the present invention is the administration of multiple agents, such as nicotine, an analog thereof or a nicotine antagonist in conjunction with a second agent such as an anti-depressant. It is contemplated that these agents may be given simultaneously, in the same formulation. It is further contemplated that the agents are administered in a separate formulation and administered concurrently, with concurrently referring to agents given within 30 minutes of each other.

In another aspect, the second agent is administered prior to administration of nicotine, an analog thereof or a nicotine antagonist. Prior administration refers to administration of the second agent within the range of up to eight hours, seven hours, six hours, five hours, four hours, three hours, two hours, or one hour prior to treatment with nicotine, up to 30 minutes before administration of the nicotine. It is further contemplated that the second agent is administered subsequent to administration of the nicotine, an analog thereof or a nicotine antagonist. Subsequent administration is meant to describe administration from 30 minutes after first application of the nicotine treatment up to one hour, two hours, three hours, four hours, five hours, six hours, seven hours or eight hours subsequent to nicotine administration.

The amounts of nicotine, analog thereof or nicotine antagonist in a given dosage of nicotine composition will vary according to the size and sex of the individual to whom the therapy is being administered, the amount of nicotine taken in if the patient was a smoker, as well as the characteristics of the disorder being treated. The method of the invention provides for administration of the composition of the invention in a therapeutically effective amount to relieve the symptoms of ulcerative colitis or alleviate the need for smoking in a chronic disease. In exemplary treatments, it may be necessary to administer up to 40 mg/day, up to 35 mg/day up to 30 mg/day, up to 25 mg/day, up to 20 mg/day, up to 15 mg/day, up to 10 mg/day, up to 5 mg/day, or up to 3 mg/day of nicotine or nicotine analog. Standard dose-response studies, in experimental animal models and in clinical testing, reveal optimal dosages for particular disease states and patient populations.

The amount of anti-depressant in a given dosage may also vary according to the size and sex of the individual being treated. For example, if the anti-depressant used in the nicotine composition is bupropion, buproprion treatment is described in U.S. Pat. No. 6,495,605, as given within a range of 10 to 750 mg. Preferably, bupropion is given in a range of 50 mg/treatment to 300 mg/treatment.

In one aspect, the invention contemplates that the composition is administered in a dose range between 5 mg/day and 40 mg/day of nicotine or nicotine analog, and 50 mg/day and 400 mg/day of bupropion. In one embodiment, the composition is administered at a dose of 15-40 mg/day nicotine or nicotine analog and 2000 mg bupropion per day. In another embodiment, the composition is administered at a dose of 15-40 mg/day nicotine or nicotine analog and 400 mg bupropion per day.

In order to treat chronic diseases, it may be necessary to administer a composition of the invention for a longer period of time that would be necessary to halt the craving for nicotine. In one aspect, the invention provides a method wherein the composition is administered to a patient for a period greater than six months. In a related aspect, the invention provides a method wherein the composition is administered to a patient for a period greater than a year.

It will also be apparent that dosing should be modified if traditional therapeutics are administered in combination with therapeutics of the invention.

Kits

As an additional aspect, the invention includes kits which comprise one or more compounds or compositions packaged in a mainer which facilitates their use to practice methods of the invention. In one aspect, the invention provides a kit comprising: (a) a pharmaceutical composition comprising a combination of nicotine, an analog thereof, or a nicotine antagonist and an anti-depressant; and (b) an indication that the pharmaceutical composition is useful for treating ulcerative colitis. In one embodiment, the anti-depressant of the kit is bupropion.

In a related embodiment, the kit includes a nicotine composition described herein (e.g., a composition comprising nicotine, an analog thereof or nicotine antagonist alone or in combination with a second agent such as an anti-depressant) packaged in a container such as a sealed bottle or vessel, with a label affixed to the container or included in the package that describes use of the composition in practicing the method. Preferably, the composition is packaged in a unit dosage form.

Preferably, the kit contains instructions, such as a product insert or label, that describes use of the nicotine composition and is useful for the treatment of the indications listed on the label or product insert. An “indication” as used herein refers to the use of a pharmaceutical composition for treating a particular disease. Indications contemplated by the present invention include inflammatory bowel disease, such as ulcerative colitis, and other chronic diseases as a smoking substitute, as set out herein.

In one embodiment the label or product insert describes how and when to administer the nicotine/anti-depressant composition. In a related embodiment, if the kit is provided with a nicotine composition separate from the anti-depressant, the label or product insert provides guidance as to when to administer the anti-depressant relative to the nicotine composition, or vice-versa. In another embodiment, the label or insert also provides a phone number or internet link for patients to consult regarding potential side-effects of the nicotine or nicotine/anti-depressant composition and methods for alleviating those side effects.

The kit may further include a device suitable for administering the composition according to a specific route of administration.

Additional aspects and details of the invention will be apparent from the following examples, which are intended to be illustrative rather than limiting.

### EXAMPLE 1

### Treatment of Colitis with a Nicotine Composition Comprising a Nicotine/Bupropion Patch

The average smoker smokes approximately 28 cigarettes per day, with each cigarette containing approximately 1-2 mg of nicotine. An appropriate dose of the nicotine, nicotine analog or a nicotine antagonist in combination with an anti-depressant is similar to the nicotine dosage obtained by the smoker from cigarettes. Alternatively, the treatment provides a gradual dose of nicotine, increasing nicotine influx to colitis patients who are non-smokers in order to make the treatment more physiological acceptable to an individual not used to nicotine intake.

In one aspect, the subject having ulcerative colitis wears a patch comprising a nicotine composition, such as nicotine, an analog thereof or a nicotine antagonist, and bupropion, wherein the nicotine patch delivers approximately 20 mgs of nicotine to the patient per 24 hour period and the amount of bupropion delivered is approximately 150 mg-400 mgs per day. For initial treatment of colitis with the nicotine composition, it is contemplated that another nicotine/bupropion patch is available at ½ the dosage given above, for example, 10 mg nicotine and 200 mg bupropion (10/200).

For patients who have not smoked for over a year or who have never smoked, to begin the colitis treatment, a patient may use a patch comprising the ½ dosage (10/200) for 30 days. The patch may be worn all day (approximately 16 hours) and removed at bedtime so as to not interfere with the patient's sleep routine. After 30 days on the ½ dosage 10/200 patch, the subject begins to use the full dosage patch, worn all day and removed at bedtime. If, after 60 days an improvement is not observed, the patient may increase the dosage by using a combination of patches and/or leaving the patch on for 24 hours. The maximum recommended dosage in any 24 hour period is 40 mg nicotine and 400 mg bupropion.

If adverse reactions such as sleeplessness, dizziness or other side effects occur, the patient may reduce the amount of time the nicotine/bupropion patch is worn, may stop using it for a few days, or if the reaction is severe, the patient may discontinue use altogether for a period of time.

Typically, a colitis patient will see improvement within 30 to 60 days. If after 90 to 120 days of wearing the patch there are no signs of improvement, another dosage regimen is attempted. Dosage requirements may also vary depending on the stress level perceived by the patient undergoing treatment.

As a treatment of ulcerative colitis in individuals who are non-smokers or smoke to ease the symptoms of colitis, a regimen whereby the amount of nicotine is gradually increased is used.

In one aspect, for the first month of treatment, 200 mg bupropion and 10 mg nicotine in combined patch form or given in the form of nicotine patch and bupropion pill. The patch is preferably taken off before going to bed. This dosage will adjust the person to the nicotine, especially if the person has not had nicotine before. After the first month, the dosage is increased to 20 mg of nicotine and 200 mg of bupropion in combined patch form or the nicotine in patch form and the bupropion in the pill form. This will cause the colitis to regress. The patch may be taken off before going to bed. If the person does not notice a reduction in the colitis symptoms after one month at the 20/200 dosage, the patch may be worn throughout the day and night.

In a related aspect, for a patient who has not smoked for over a year, or stopped smoking within a year, a patient may use a nicotine composition patch or combination of patches wherein the mgs of nicotine match the number of cigarettes the patient used to smoke in a day. For example, if a patient typically smoked one pack of cigarettes a day, the patient may start the treatment using the 20/200 treatment, or two 10/200 patches, depending on the dose of bupropion desired. The maximum dosage should not exceed two 20/200 patches over a 24 hour period. After the colitis goes into remission, the patient may reduce the nicotine/bupropion treatment to a minimum dosage level required to maintain the remission state.

Treatment efficacy is assessed using disease activity indexes and physiological measurements as described herein. Nicotine composition therapy that reduces at least one symptom of ulcerative colitis, such as rectal bleeding or inflammation, and manifests as an improvement in activity index score indicates that the combination of nicotine and bupropion is an effective treatment for ulcerative colitis.

It is also contemplated that the above treatment regimens are administered to ulcerative colitis patients undergoing more traditional therapies such as treatment with corticosteroids, aminosalicylates, steroid hormones or probiotic therapies. For example, colitis sufferers receiving mesalazine, salazosulfapyridine or budesonide therapy may be administered an amount of nicotine composition such as the ½ dosage patch that may be suboptimal if given alone, but may act in conjunction with the other treatment to potentiate the effects of the traditional therapy. As used herein, “potentiate” or “synergize” refers to a nicotine composition, which, when given in conjunction with either a traditional therapy for ulcerative colitis, enhances the therapeutic effect of the treatment beyond that of administration of the nicotine composition or the traditional therapy alone.

It is expected that treatment with the nicotine composition in combination with other therapies will produce greater relief to the patient than a single treatment regimen alone.

### EXAMPLE 2

### Use of Nicotine/Bupropion Patch as a Long Term Substitute For Smoking

As a general categorization, there are three classes of smokers: a) those that want to quit smoking, b) those that want to reduce their smoking, and c) those that want to substitute the nicotine/bupropion patch for smoking. The method of the invention utilizing the composition as a substitute for smoking may be used for patients undergoing treatment for diseases such as emphysema, heart conditions, high blood pressure, or treatment for cancer, in which smoking during treatment, or any sort of smoking whatsoever, is prohibited.

For example, if the colitis patient or patient with a heart condition to be treated is a habitual smoker who requires the act of smoking but cannot smoke, or cannot smoke their typical number of cigarettes per day due to a medical reason, a modified patch regimen is used. For example, if a patient typically smoked 40 cigaretettes a day, the patient may wear a 20/200 patch, or wear two 10/200 patches and smoke 20 cigarettes. Alternatively, the patient may smoke 5-10 cigarettes a day, and wear a 10/200 patch 8 hours/day; smoke 10-14 cigarettes a day, and wear a 10/200 patch 16 hours a day; smoke 15-19 cigarettes a day and wear a 10/400 patch; smoke 20-25 cigarettes a day, wear a 10/400 mg bupropion patch; or smoke 30 or more cigarettes a day, wear the 10/400 mg bupropion patch 24 hours a day. Any combination of smoking and patch dosage is available that allows the patient to function and ameliorates disease symptoms. The goal of the dosage is to make sure the smoker does not get more nicotine from the nicotine/bupropion patch than from a typical day of cigarette smoking (assuming 1 mg of nicotine per cigarette.)

In one aspect, the treatment may begin wherein for the first month, the patient wears a patch containing 200 mg of bupropion and 10 mg of nicotine, and reduces the cigarettes by one third and wears the combination nicotine/bniproion patch for 16 hours (e.g., taken off at bedtime). The patch may be worn for 24 hours, but the patient should cut the cigarettes intake accordingly. For the second month, the dosage is increased such that 200-400 mg of bupropion and 20 mg of nicotine are absorbed per day, either using a 20/200 patch or a combination of 10/200 patches, and the cigarettes intake reduced by two thirds. This patch is worn for 16 hours (taken off at bedtime). This combination patch may be left on for 24 hours, but then the patient should stop smoking altogether.

The object is that as the patient increases their nicotine/bupropion dosage, the number of cigarettes smoked is reduced. The end goal is to have the patient cease smoking and use the lowest doage of nicotine/bupropion possible to alleviate disease symptoms. A patient's dose should not exceed 40 mg of nicotine per day, using any combination of cigarette and nicotine patch relief, or 400 mg bupropion.

It is contemplated that the same type of treatment regimen described above is used to reduce the need for cigarettes in patients undergoing treatment for other diseases, such as emphysema, heart conditions, high blood pressure, or treatment for cancer. A patient in need of reducing of smoking or nicotine intake without the act of smoking is treated with the nicotine/bupropion patch as described above to alleviate the desire to smoke while undergoing a lifetime of treatment for a chronic disease. The therapy described herein is designed for the longterm use of the nicotine patch, rather than a short term burst of nicotine to provide temporary relief from the desire to smoke.

For all treatments described above, the patch is worn on the sides of the body on the trunk area, not on the arms or below the waist. If, at any time in the treatment the patient feels numbness in the arms or neck, the patch may be removed, and the dosage reduced by not wearing the patch as long the next day.

Numerous modifications and variations in the invention as set forth in the above illustrative examples are expected to occur to those skilled in the art. Consequently only such limitations as appear in the appended claims should be placed on the invention.

1. A method for treating a patient suffering from ulcerative colitis comprising administering a pharmaceutical composition comprising a combination of nicotine, an analog thereof or a nicotine antagonist, and an anti-depressant, wherein the pharmaceutical composition is administered in an amount effective to reduce the symptoms of ulcerative colitis.

2. The method of claim 1 wherein the anti-depressant is bupropion.

3. The method of claim 1 wherein the composition is administered transdermally.

4. The method of claim 1 wherein the composition is administered orally.

5. The method of claim 1 wherein the nicotine composition and anti-depressant are given in conjunction with an additional ulcerative colitis treatment selected from the group consisting of an aminosalicylate, a corticosteroid, an immunomodulatory medicine, a steroid hormone, and a probiotic therapy.

6. The method of claim 5 wherein the ulcerative colitis treatment is mesalazine, salazosulfapyridine, or budesonide.

7. The method of claim 1 wherein the composition is administered to a patient for a period greater than six months.

8. A substitute for smoking comprising administering to a subject for a period greater than two months a composition comprising nicotine, or an analog thereof, and an anti-depressant.

9. The method of claim 8 wherein the subject exhibits a condition selected from the group consisting of inflammatory bowel disease, emphysema, chronic heart failure, lung cancer, and esophageal cancer.

10. The method of claim 8 wherein the composition is administered transdermally.

11. The method of claim 8 wherein the composition is administered orally.

12. The method of claim 8 wherein the composition is administered to a patient for a period greater than six months.

13. The method of claim 12 wherein the composition is administered to a patient for a period greater than a year.

14. The method of any one of claims 1-13 wherein the composition is administered in a dose range between 5 mg/day and 40 mg/day of nicotine and 50 mg/day and 400 mg/day of bupropion.

15. The method of claim 14 wherein the composition is administered at a dose of 10-40 mg/day nicotine and 200 mg bupropion.

16. The method of claim 14 wherein the composition is administered at a dose of 10-40 mg/day nicotine and 400 mg bupropion.

17. A kit comprising:
(a) a pharmaceutical composition comprising a combination of nicotine, a nicotine analog or nicotine antagonist, and an anti-depressant; and
(b) an indication that the pharmaceutical composition is useful for treating ulcerative colitis.

18. The kit of claim 17 wherein the anti-depressant is bupropion.
